(19) United States
(12) Patent Application Publication
(10) Pub. No.: US 2009/0318568 A1
(43) Pub. Date: Dec. 24, 2009

(54) ADHERENT COATING FOR TISSUE SURFACE AND/OR TRANS-TISSUE SURFACE SUBSTANCE DELIVERY

(76) Inventor: Ray L. Hauser, Boulder, CO (US)

Correspondence Address: COCHRAN FREUND & YOUNG LLC, 2026 CARIBOU DR, SUITE 201, FORT COLLINS, CO, 80525, US

(21) Appl. No.: 12/548,336

(22) Filed: Aug. 26, 2009

(51) Int. Cl. A61K 47/32; A61P 35/00

(52) U.S. Cl. 514/772.5; 523/105

(57) ABSTRACT

An adherent coating composition is described having a reservoir of medication or other substance and capable of intimate contact with a target area of exposed tissue, rapid conveyance of the medication onto or into the target area. Mixtures of polyvinylpyrrolidone and glycerin have been found to dissolve a large number of medications while producing an adherent film. Application of the present composition to provide anti-cancer medication to exposed tissue resulting from tumor resection is also described.

### FIELD OF THE INVENTION

The present invention relates generally to infusion of medication into a patient and, more particularly, to an adherent coating effective for tissue surface and/or trans-tissue surface delivery of medication or other substances to a patient.

### BACKGROUND OF THE INVENTION

There are 170,000 new cases of lung cancer per year. There has been no effective procedure identified for post-surgical treatment of residual cancer cells left in the patient after tumor resection. Paclitaxel (Taxol) has been applied to surfaces exposed during surgery, but it has been found not to remain in place sufficiently long to generate beneficial effects.

The use of transdermal compositions for controlling medicament delivery through the skin at a substantially constant rate is known. Such delivery systems may incorporate a medicament into a carrier such as a polymeric matrix, as an example, which effectively adheres to the skin, thereby permitting infusion of the medicament from the carrier through the skin and into the bloodstream of a patient. Self-supporting polymeric diffusion matrices comprising a polar plasticizer, polyvinyl alcohol, and polyvinylpyrrolidone, for the sustained release of a pharmaceutically effective amount of propranolol, phenylephrine, clonidine, terbutaline, and phenylpropanolamine contained therein, respectively, by transdermal delivery to a patient have been described.

A pharmaceutical composition has been described that includes hyaluronate and polyvinylpyrrolidone in the form of a membranous, wafer-like material after being lyophilized. Other ingredients may be added before lyophilization, depending on the intended use, include maltodextrin, hydroxyethylcellulose, glycerin, human cellular fibronectin, and antimicrobial agents, as examples. The resulting material, after lyophilization, is a light, fluffy, white membrane or wafer-like material. When the composition is placed on a wound, it absorbs moisture and adheres tightly to the surface. The formulation may be used in liquid form by mixing the dried material (wafer-like or powder) with water. Cellular fibronectin is used to coat cut surfaces of tissues following surgery for cancer to decrease the incidence of local recurrence of the malignancy.

### SUMMARY OF THE INVENTION

Accordingly, it is an object of the present invention to provide an adherent composition having a reservoir of medication for application to skin and to tissue surfaces exposed during surgery.

Another object of the invention is to provide an adherent sprayable or paintable composition having a reservoir of at least one substance for application to skin and to tissue surfaces exposed during surgery.

Yet another object of the invention is to provide an adherent composition having a reservoir of at least one substance for application to skin and to tissue surfaces exposed during surgery, and to provide a paintable or sprayable non-sticky composition effective as a barrier coating over the adherent composition.

Still another object of the invention is to provide an adherent composition having a reservoir of anti-cancer medication for application to tissue surfaces exposed during resection of a tumor.

Another object of the invention is to provide a colored, paintable or sprayable adherent composition having a reservoir of at least one substance to skin and to tissue surfaces exposed by surgery such that the area coated and the coating thickness may be made apparent.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

To achieve the foregoing and other objects, and in accordance with the purposes of the present invention, as embodied and broadly described herein, the composition hereof, includes: polyvinylpyrrolidone as the principal adherent polymeric material, a polar plasticizer, a substance soluble in the plasticizer to be delivered onto a skin surface or other exposed tissue, or delivered trans-surface thereto, and a solvent therefor.

In another aspect of the invention, and in accordance with its objects and purposes, the composition hereof includes: polyvinylpyrrolidone, at least one polar plasticizer, at least one substance to be delivered onto a skin surface or other exposed tissue or delivered trans-surface thereto, and a solvent therefor, in the absence of polyvinyl alcohol in amounts greater than or equal to 2 wt. %.

In yet another aspect of the invention, and in accordance with its objects and purposes, the composition hereof consisting essentially of: polyvinylpyrrolidone, at least one polar plasticizer, at least one substance to be delivered onto a skin surface or other exposed tissue or delivered trans-surface thereto, and a solvent therefor.

In still another aspect the present invention, and in accordance with its objects and purposes, the method for delivering at least one chosen substance onto an exposed tissue surface or trans-surface thereto, hereof, includes the steps of: providing a composition including polyvinylpyrrolidone as the principal adherent polymeric material, at least one polar plasticizer, the at least one chosen substance, and a solvent therefor; and applying the composition to the surface such that an adherent film is formed thereon.

In another aspect of the present invention, and in accordance with its objects and purposes the method for delivering at least one chosen substance to an exposed tissue surface or trans-surface thereto, hereof, includes the steps of: providing a composition including polyvinylpyrrolidone, at least one polar plasticizer, the at least one chosen substance, and a solvent therefor, in the absence of polyvinyl alcohol in amounts greater than or equal to 2 wt. %; and applying the composition to the surface such that an adherent film is formed thereon.

In yet another aspect of the present invention, and in accordance with its objects and purposes the method for delivering at least one chosen substance to an exposed tissue surface or trans-surface thereto, hereof, includes the steps of: providing a composition consisting essentially of: polyvinylpyrrolidone, at least one polar plasticizer, the at least one chosen substance, and a solvent therefor; and applying the composition to the surface such that an adherent film is formed thereon.

In another aspect of the present invention, and in accordance with its objects and purposes, the method for delivering an anti-cancer chemotherapeutic agent to the surface of tissue exposed after removal of a malignant tumor and/or trans-surface thereto, hereof, includes the steps of: providing a composition including: polyvinylpyrrolidone, at least one polar plasticizer, at least one anti-cancer chemotherapeutic agent, and a solvent therefor; and applying the composition to the surface such that an adherent film is formed thereon.

Benefits and advantages of the present invention include, but are not limited to, providing a paintable (brushable) and/or sprayable composition having a reservoir of medication and capable of intimate contact with a target area of tissue, rapid conveyance of the medication to the target area, and effective for forming a flexible film adherent to the tissue for a sufficient period or time such that the medication can be conveyed thereto. With the addition of a colorant, the extent of coverage and an estimate of the amount of coating delivered may be obtained. The application of a second coating which may derive from a solution of polyvinylpyrrolidone in alcohol or water or a solution polyvinyl alcohol in alcohol or water, or a mixture of the two solutions over the first adherent coating may beneficially minimize the external tackiness of the first coating.

### DETAILED DESCRIPTION OF THE INVENTION

Briefly, the present invention includes a composition of matter including polyvinylpyrrolidone (PVP), a polar plasticizer or a mixture of polar plasticizers, such as glycerol, propylene glycol, and propylene carbonate, an effective amount of medication or other substance, and a solvent therefor, for forming a paintable (brushable) or sprayable composition. The composition forms a flexible adherent film such that the medication or other substance may be disposed in intimate contact with the target area of the body which may be skin or tissue exposed during surgery, whereby the medication or other substance is conveyed to the target by diffusion of the polar plasticizer. The composition is capable of sticking to wet tissue since, as the solvent evaporates, the viscosity of the composition significantly increases. Small amounts of polyvinyl alcohol may be used in the composition of matter hereof (less than 2 wt. %), but the PVP is utilized as the major film-forming adherent material. The composition serves as a reservoir of medication which is dissolved in the plasticizer(s), and the adherent film has sufficient thickness, flexibility and strength that it is expected to remain in place for delivery of medications over at least a 24-hour period.

It is anticipated that the application of chemotherapeutic, antibiotic and antifungal substances in accordance with the teachings of the present invention will prevent re-growth and lymphatic migration of residual malignant cells, as well as providing protection against infection for the open tissues. Moreover, a minute amount of the oncological treatment is anticipated to be effective when applied topically after resection, in comparison to whole-body doses applied intravenously, which may be in the range between 20 and 160 mg/Kg of body weight. Thus, toxic and side effects should be minimal during the time when a patient is recovering from a tumor resection.

In some embodiments of the invention, a second coating which includes a solution of a water-soluble polymer that can be resorbed into the body may be applied. Such solutions may include a solution of PVP in an alcohol, such as ethanol for rapid evaporation or isopropanol, or water, or a solution of polyvinyl alcohol (PVA) in water, may be applied to the first adherent coating in order to reduce the surface tackiness thereof. Mixtures of slow and rapid evaporating solvents may be used. The second layer bonds to the primary medicated coating due to its similarity of polymeric composition or solvency, but prevents undesirable tackiness that may cause body parts to undesirably bond to one another when the surgical cavity is open and during its closure. Both the first and second layers may further include colorants to permit the ready determination of the extent of application of the film and an estimate of its thickness. As examples, for contrast with a white primary coating, the second coating may include a green or blue color from medically acceptable coloring agents. A dye, such as green food coloring may be used in the primary coating and a white coloration may be used in the secondary coating. The second coating may optionally include an antiseptic such as iodine or bacitracin, as examples.

Typical thickness for each coating are a few hundred microns.

Polyvinylpyrrolidone (Chemical Abstracts Numbers 9003-39-8, 9080-59-5, and 84057-81-8) is a thermoplastic that is self-tackifying when dissolved in selected solvents. The terms “polyvinylpyrrolidone” or PVP, as used herein, refer to a polymer, either a homopolymer or copolymer, containing N-vinylpyrrolidone as the monomeric unit. Typical PVP polymers are homopolymeric PVPs known in the pharmaceutical industry under a variety of designations including Povidone, Polyvidone, Polyvidonum soluble, and Poly(1-vinyl-2-pyrrolidone). The term “soluble” when used with reference to PVP means that the polymer is soluble in water and/or alcohols and is generally not substantially cross-linked.

As used herein, PVP also includes copolymers of polyvinylpyrrolidone and mixtures of this polymer with other water and/or alcohol-soluble polymers such as poly(ethyl oxazoline), polyhydroxy ether, poly(ethylene oxide), poly(ethenyl formamide), and polyvinyl alcohol, as examples.

A relatively nonvolatile solvent can dissolve the PVP as a plasticizer to give the desired adhesive physical properties which are dependent upon the ratio of solid and liquid. PVP is available in molecular weights ranging between about 10,000 and about 1.3 million Daltons, the higher molecular weights yielding stronger, stiffer adhesives. PVP has a solubility parameter about 11.4 [cal/cc]^{1/2} (2.045 [cal/cc]^{1/2}=MPa^{0.5}) and it is readily soluble in polar solvents such as water, alcohols, polyols, and alkyl carbonates. The water resistance of PVP can be increased by partially cross-linking this polymer by radiation or chemical means before or after dissolution in the plasticizer/solvent.

One suitable plasticizer, glycerol (also known as glycerin), has a solubility parameter of about 16.5 [cal/cc]^{1/2}. The PVP/glycerol formulation of the present invention inherently has a high rate of permeation by virtue of the glycerol content. It needs no further enhancement for use on water-absorbing tissues, but may be enhanced for application to fatty tissues. Alternative polar plasticizers having solubility parameters greater than 10 [cal/cc]^{0.5}, and boiling temperatures above 150° C. may be used. Propylene glycol and propylene carbonate are effective plasticizer/solvents for PVP.

The term “substance” as used herein includes adjuvants, drugs and biologicals, such as cancer treatment drugs, as examples. Barrier pigments may include flaky materials having large surface areas in relation to their thickness dimensions.

Taxanes are modifications of paclitaxel or docetaxel made by addition of carriers, chelates, or by chemical reaction. These include paclitaxel plus albumin (for example, Abraxane), PG paclitaxel (polyglutamate adduct), DHA-paclitaxel (docosahexanoic acid adduct known as Taxoprexin®), PEG paclitaxel (polyethylene glycol adduct), and Tumor-Activated Prodrugs based on paclitaxel, as examples, are among the taxanes appropriate for the present topical application.

PVP and glycerol are GRAS (Generally Recognized as Safe) chemicals. PVP has been used as a component of blood plasma. Glycerol has been used as an internal medication and as a component of many cosmetics for many years. As stated hereinabove, glycerol is used both as a plasticizer for the PVP and as a solvent and mobile medium for transferring the medication through the skin.

Reference will now be made in detail to the present embodiments of the invention. A medicated coating using PVP and glycerol is inherently permeable to mass transfer of water therethrough. The adherent coating is expected to be compatible with the tissues on which it is applied, and to remain in place by virtue of its good adhesion and flexible nature for multiple days.

A treatment for exposed tissues after tumor resection may include spraying a solution of polyvinylpyrrolidone, glycerol, and one or more anti-tumor medications, such as paclitaxel, cisplatin, doxorubicin, curcumin and taxanes, as examples, and a coloring agent, such as zinc oxide, titanium dioxide, or a food dye or colorant, as examples, to provide a visual confirmation of coverage area and thickness. Barrier pigments, such as talc, mica or aluminum flake may be used. The treatment may also include lipophilic and/or amphiphilic agents, such as surfactants (for example, Triton X-100), fatty acids and monoglycerides, as examples. Combination of the present compositions with aqueous or alcohol solution to form a sprayable or brushable mixture effective for application to all (including wet) exposed tissue surfaces and bonding thereto. A second spray of a non-sticky barrier coating may then be applied; such coating may include polyvinylpyrrolidone, iodine, talc, colorant and solvent. Other components of the present composition may include isotonic materials such as sugars or saline and/or antibacterial and antifungal agents such as parabens, chlorbutanol, phenol, sorbic acid and/or thimerosal.

The dissolution of PVP in glycerols may be accomplished by gradual addition of the PVP powder to the glycerol/alcohol solution with stirring. Warming can facilitate complete dissolution which is evidenced by clarity of solution. Medications or other substances may be added to the glycerol/alcohol solution before or after making the PVP solution. Anti-tumor medications such as paclitaxel, cisplatin, doxorubicin, curcumin, and taxanes are known to be soluble in alcohols in low concentrations appropriate for direct application to tissues, and are expected to be soluble in glycerol and propylene glycol at effective concentrations. Addition of other components such as isopropyl myristate, albumin, cremophors, cyclodextrin at a concentration between 1% and 20% by weight, and randomly methylated-β-cyclodextrin at a concentration between 1% and 20% by weight, as examples, are known to assist solubility, and additions of small amounts of acetic acid (at concentrations between 0.1% and 0.5% by weight) are known to assist in the stabilization of taxanes in solution.

Mixtures of multiple anti-tumor medications are known to be more effective than a single medication alone. The selection of such components may be specific to the type of cancer being treated.

Having generally described the invention, the following EXAMPLES provide additional details:

### EXAMPLE 1

To 100 g of USP glycerol warmed to about 50° C., 100 mg of paclitaxel (Paxis Pharmaceuticals, Boulder, Colo.) was added; however, but little of the paclitaxel appeared to dissolve. To this mixture, 60 g of 1,2-propylene glycol was added; the new mixture was mixed, and warmed further. The undissolved solids were filtered off, and the remaining solution was analyzed and found to contain 0.276 mg/ml. of taxanes, including 0.239 mg/ml of paclitaxel.

This solution was used to prepare a sprayable coating having the following formula:
Taxane solution of glycerol+propylene glycol+paclitaxel: 1 g;
190-proof alcohol: 25 ml, 19.7 g;
99% isopropanol: 25 ml, 19.5 g;
Polyvinylpyrrolidone: 1.3M Daltons, 2.5 g;
Triton X-100: 0.25 ml; and
Green food coloring: 1 ml.
   This coating was sprayed onto a sheet of dialysis-grade cellophane having an area of 237 cm² for 30 s, giving a clearly discernible green coloration estimated to have a thickness of about 125 μm. A barrier, over-coat was prepared having the following formula:
Polyvinylpyrollidone, 1.3M Daltons: 2.5 g;
Glycerol: 1 g;
190-proof alcohol: 37 ml, 20 g;
Mistron RCS talc: 3.5 g for coloration and for decreasing the tackiness of the barrier film; and
Triton X-100: 0.25 ml, for decreasing the tackiness of the barrier film.
   This coating was sprayed over the green coating and formed a non-tacky, color contrast second coating, estimated to have a thickness of about 125 μm.

The cellophane was placed in a chamber having approximately 100% relative humidity at about 40° C. for 24 h, and then washed with water to remove the surface coatings. The cellophane was then extracted using methanol and a Soxhlet apparatus, and the extract was analyzed by gas chromatography for taxanes. The cellophane was found to contain 0.142 mg of taxanes, or 0.60 μg/cm², which confirmed the ability of the glycerol/glycol solution to transport the taxanes into a cellulose membrane.

### EXAMPLE 2

The residue from filtering the glycerol solution of EXAMPLE 1 was added to 100 g of 1,2-propylene glycol, and the resulting mixture warmed to 50° C.; little of the solid material appeared to dissolve. The mixture was then placed in an oven at 135° C. and heated for 45 min. All of the taxanes dissolved in the glycol, and analysis showed 0.23 mg/ml of paclitaxel in solution, of 0.460 mg/ml total taxanes.

This solution was used to prepare a sprayable coating having the formula:
Taxane solution of paclitaxel in propylene glycol: 1 g;
190-proof alcohol: 25, ml, 19.7 g;
99% isopropanol, 25 ml: 19.5 g;
Polyvinylpyrrolidone, 1.3M Daltons: 2.5 g;
Triton X-100: 0.25 ml; and
Green food coloring: 1 ml.
   This coating was sprayed onto a sheet of dialysis-grade cellophane having an area of 237 cm² for 30 s, giving a clearly discernible green coloration estimated to have a thickness of about 125 μm. The white, non-tacky barrier coating of EXAMPLE 1, hereof, was sprayed over the green medicated coating, and the coated cellophane was treated similarly to that in EXAMPLE 1. Soxhlet extraction and chromatographic analysis showed 0.148 mg of taxanes or 0.62 μg/cm².

### EXAMPLE 3

A white mixture was prepared for spraying onto dark tissues to investigate color contrasts and to investigate the use of non-flammable coatings:
Glycerol: 5 g
Polyvinylpyrrolidone, 1.3 M Daltons: 5 g;
Water: 100 ml; and
Zinc oxide pigment: 10 g
   This mixture was sprayed for 40 s onto approximately 100 cm² of beef liver. The coating had good color contrast, but it dried more slowly than did the coatings of EXAMPLES 1 and 2, hereinabove, and a hair dryer on low setting was used to achieve moderate drying, which was limited by the wetness of the liver. A green overcoat having the formula:
Glycerol: 1 g;
Polyvinylpyrrolidone, 1.3 M Daltons: 2.5 g;
190 proof alcohol: 37 ml;
99% isopropanol: 17 g;
Green food coloring: 1 ml;
Triton X-100: 0.25 ml for decreasing the tackiness of the barrier film; and
Mistron talc: 3.5 g for decreasing the tackiness of the barrier film.
   Good color contrasts were observed, and the new surface was relatively non-tacky.

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A composition of matter comprising: polyvinylpyrrolidone as the principal adherent polymeric material, at least one polar plasticizer, at least one substance soluble in the plasticizer to be delivered onto skin or other exposed tissue, or delivered trans-surface thereto, and a solvent therefor.

2. The composition of claim 1, further comprising a coloring agent.

3. The composition of claim 1, wherein the at least one plasticizer comprises plasticizers having solubility parameters greater than 10 [cal/cc]^{0.5}, and boiling temperatures above 150° C.

4. The composition of claim 3, wherein the at least one plasticizer is chosen from glycerol, propylene glycol and propylene carbonate.

5. The composition of claim 1, wherein the solvent is chosen from water, ethanol, and isopropyl alcohol, and mixtures thereof.

6. The composition of claim 1, wherein the at least one substance is chosen from adjuvants, drugs and biologicals.

7. The composition of claim 1, wherein the at least one substance comprises at least one chemotherapeutic, anti-cancer agent.

8. A composition of matter comprising: polyvinylpyrrolidone, at least one polar plasticizer, at least one substance to be delivered onto skin or other exposed tissue or delivered trans-surface thereto, and a solvent therefor, in the absence of polyvinyl alcohol in amounts greater than or equal to 2 wt. %.

9. The composition of claim 8, further comprising a coloring agent.

10. The composition of claim 8, wherein the at least one plasticizer comprises plasticizers having solubility parameters greater than 10 [cal/cc]^{0.5}, and boiling temperatures above 150° C.

11. The composition of claim 10, wherein the at least one plasticizer is chosen from glycerol, propylene glycol and propylene carbonate.

12. The composition of claim 8, wherein the solvent is chosen from water, ethanol, and isopropyl alcohol, and mixtures thereof.

13. The composition of claim 8, wherein the at least one substance is chosen from adjuvants, drugs and biologicals.

14. The composition of claim 8, wherein the at least one substance comprises at least one chemotherapeutic, anti-cancer agent.

15. A composition of matter consisting essentially of: polyvinylpyrrolidone, at least one polar plasticizer, at least one substance to be delivered onto skin or other exposed tissue or delivered trans-surface thereto, and a solvent therefor.

16. The composition of claim 15, wherein the at least one plasticizer comprises plasticizers having solubility parameters greater than 10 [cal/cc]^{0.5}, and boiling temperatures above 150° C.

17. The composition of claim 16, wherein the at least one plasticizer is chosen from glycerol, propylene glycol and propylene carbonate.

18. The composition of claim 15, wherein the solvent is chosen from water, ethanol, and isopropyl alcohol, and mixtures thereof.

19. The composition of claim 15, wherein the at least one substance comprises is chosen from adjuvants, drugs and biologicals.

20. The composition of claim 15, wherein the at least one substance comprises at least one chemotherapeutic, anti-cancer agent.

21. A method for delivering at least one chosen substance to an exposed tissue surface or trans-surface thereto, comprising the steps of: providing a first composition comprising: polyvinylpyrrolidone as the principal adherent polymeric material, at least one polar plasticizer, the at least one chosen substance, and a first solvent therefor; and applying the first composition to the surface such that a first adherent film is formed thereon.

22. The method of claim 21, wherein the first composition further comprises a first coloring agent.

23. The method of claim 21, further comprising the steps of: providing a second composition comprising: polyvinylpyrrolidone, glycerol, and a second solvent therefor; and applying the second composition to the first film, whereby a barrier film is formed on the first film.

24. The method of claim 23, wherein the second composition further comprises a second coloring agent.

25. The method of claim 24, wherein the second coloring agent is chosen from zinc oxide, titanium dioxide, barrier pigments and food colorants.

26. The method of claim 25, wherein the barrier pigments are chosen from talc, mica and aluminum flake.

27. The method of claim 21, wherein the at least one plasticizer comprises plasticizers having solubility parameters greater than 10 [cal/cc]^{0.5}, and boiling temperatures above 150° C.

28. The method of claim 27, wherein the at least one plasticizer is chosen from glycerol, propylene glycol and propylene carbonate.

29. The method of claim 21, wherein the first solvent is chosen from water, ethanol, and isopropyl alcohol, and mixtures thereof.

30. The method of claim 21, wherein the at least one substance is chosen from adjuvants, drugs and biologicals.

31. The method of claim 21, wherein the at least one substance comprises at least one chemotherapeutic, anti-cancer agent.

32. A method for delivering at least one chosen substance to an exposed tissue surface or trans-surface thereto, comprising the steps of: providing a first composition comprising polyvinylpyrrolidone, at least one polar plasticizer, the at least one chosen substance, and a first solvent therefor, in the absence of polyvinyl alcohol in amounts greater than or equal to 2 wt. %; and applying the first composition to the surface such that an adherent film is formed thereon.

33. The method of claim 32, wherein the first composition further comprises a first coloring agent.

34. The method of claim 32, further comprising the steps of: providing a second composition comprising: polyvinylpyrrolidone, glycerol, and a second solvent therefor; and applying the second composition of matter to the first film, whereby a barrier film is formed on the first film.

35. The method of claim 34, wherein the second composition further comprises a second coloring agent.

36. The method of claim 35, wherein the second coloring agent is chosen from zinc oxide, titanium dioxide, barrier pigments and food colorants.

37. The method of claim 36, wherein the barrier pigments are chosen from talc, mica and aluminum flake.

38. The method of claim 32, wherein the at least one plasticizer comprises plasticizers having solubility parameters greater than 10 [cal/cc]^{0.5}, and boiling temperatures above 150° C.

39. The method of claim 38, wherein the at least one plasticizer is chosen from glycerol, propylene glycol and propylene carbonate.

40. The method of claim 32, wherein the solvent is chosen from water, ethanol, and isopropyl alcohol, and mixtures thereof.

41. The method of claim 32, wherein the at least one substance is chosen from adjuvants, drugs and biologicals.

42. The method of claim 32, wherein the at least one substance comprises at least one chemotherapeutic, anti-cancer agent.

43. A method for delivering at least one chosen substance to an exposed tissue surface or trans-surface thereto, comprising the steps of: providing a first composition consisting essentially of: polyvinylpyrrolidone, at least one polar plasticizer, at least one chosen substance, and a first solvent therefor; and applying the first composition to the surface such that an adherent film is formed thereon.

44. The method of claim 43, wherein the first composition further comprises a first coloring agent.

45. The method of claim 43, further comprising the steps of: providing a second composition comprising: polyvinylpyrrolidone, glycerol, and a second solvent therefor; and applying the second composition of matter to the first film, whereby a barrier film is formed on the first film.

46. The method of claim 45, wherein the second composition further comprises a second coloring agent.

47. The method of claim 46, wherein the second coloring agent is chosen from zinc oxide, titanium dioxide, barrier pigments and food colorants.

48. The method of claim 47, wherein the barrier pigments are chosen from talc, mica and aluminum flake.

49. The method of claim 43, wherein the at least one plasticizer comprises plasticizers having solubility parameters greater than 10 [cal/cc]^{0.5}, and boiling temperatures above 150° C.

50. The method of claim 49, wherein the at least one plasticizer is chosen from glycerol, propylene glycol and propylene carbonate.

51. The method of claim 43, wherein the first solvent is chosen from water, ethanol, and isopropyl alcohol, and mixtures thereof.

52. The method of claim 43, wherein the at least one substance is chosen from adjuvants, drugs and biologicals.

53. The method of claim 43, wherein the at least one substance comprises at least one chemotherapeutic, anti-cancer agent.

54. A method for delivering an anti-cancer chemotherapeutic agent to the surface of tissue exposed after removal of a malignant tumor or trans-tissue surface thereto, comprising the steps of: providing a first composition comprising: polyvinylpyrrolidone, at least one polar plasticizer, at least one anti-cancer chemotherapeutic agent, and a first solvent therefor; and applying the first composition to the surface such that a first adherent film is formed thereon.

55. The method of claim 54, further comprising the steps of: providing a second composition comprising: polyvinylpyrrolidone, glycerol, and a second solvent therefor; and applying the second composition to the first film, whereby a non-tacky barrier film is formed thereon.
